(19) United States
(12) Patent Application Publication
(10) Pub. No.: US 2025/0320207 A1
(43) Pub. Date: Oct. 16, 2025

(54) Ketal Protected Intermediate for Sonrotoclax and Preparation Method Thereof

(71) Applicant: BeOne Medicines I GmbH, Basel (CH)

(72) Inventors: Zhengfeng Li, Suzhou (CN); Tao Zhang, Suzhou (CN); Liwen Xu, Suzhou (CN); Juntao Liu, Suzhou (CN); Qun Jiang, Suzhou (CN)

(73) Assignee: BeOne Medicines I GmbH, Basel (CH)

(21) Appl. No.: 19/250,658

(22) Filed: Jun. 26, 2025

Related U.S. Application Data
(63) Continuation of application No. PCT/CN2023/141941, filed on Dec. 26, 2023.

(30) Foreign Application Priority Data
Dec. 27, 2022 (WO) PCTCN2022142312

(51) Int. Cl. C07D 471/04; C07D 221/20; C07D 491/113; C07D 519/00

(52) U.S. Cl. CPC C07D 471/04; C07D 221/20; C07D 491/113; C07D 519/00

(57) ABSTRACT

Disclosed herein are compounds of Formula (I) having protective group of ketal used as intermediates for preparing 2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (Sonrotoclax), and processes for preparing compounds of Formula (I), especially 1,5-dioxa-11-azadispiro[5.1.5.1]tetradecane.

### CROSS-REFERENCE TO RELATED APPLICATIONS

This application is a continuation of International Application No. PCT/CN2023/141941, filed on Dec. 26, 2023, which claims priority to International Application No. PCT/CN2022/142312, filed on Dec. 27, 2022, the disclosures of each of which are hereby incorporated by reference in their entireties.

### FIELD

Disclosed herein are compounds of Formula (I) having a protective group of a ketal used as intermediates for preparing 2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (Sonrotoclax), and processes for preparing compounds of Formula (I), especially 1,5-dioxa-11-azadispiro[5.1.5.1]tetradecane.

### BACKGROUND

International publication WO2019/210828 disclosed a series of Bcl-2 inhibitors, in particular, 2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (hereinafter Sonrotoclax), which selectively inhibit Bcl-2 proteins for the treatment of dysregulated apoptotic diseases such as cancers, autoimmune diseases and pro-thrombotic conditions.

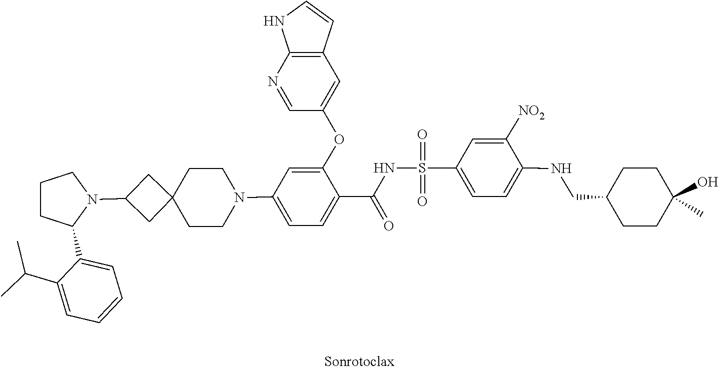 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

In the synthesis disclosed in WO2019/210828, protecting the ketone in the intermediate as 2,2-dimethoxy-7-azaspiro[3.5]nonane hydrogen chloride resulted in impurities which could not be well depleted in downstream steps. There exists a need for optimizing the synthesis of Sonrotoclax for improved impurity depletion and high stability.

### SUMMARY

Provided herein are 7-azaspiro[3.5]nonan-2-one compounds, and derivatives thereof. In some embodiments, the 7-azaspiro[3.5]nonan-2-one compound is 2,2-diethoxy-7-azaspiro[3.5]nonane, 2,2-diisopropoxy-7-azaspiro[3.5]nonane, 1,4-dioxa-10-azadispiro[4.1.5.1]tridecane, or 1,5-dioxa-11-azadispiro[5.1.5.1]tetradecane. Further provided herein are methods of making the 7-azaspiro[3.5]nonan-2-one compounds, and derivatives thereof. Further provided herein are methods of making Sonrotoclax from the 7-azaspiro[3.5]nonan-2-one compounds, and derivatives thereof.

### DETAILED DESCRIPTION

### Definitions

The following terms have the indicated meanings throughout the specification:

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include-methyl, -ethyl, -n-propyl, -n-butyl, -n-pentyl and -n-hexyl; while saturated branched alkyls include-isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include-vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

An “alkynyl” group refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2 (3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1 (2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3—, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1 (2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl. A “halogen” is fluorine, chlorine, bromine or iodine.

A “hydroxyalkyl” group is an alkyl group as described above substituted with one or more hydroxy groups.

An “alkoxy” or “alkoxyl” group is-O-(alkyl), wherein alkyl is defined above.

An “alkoxyalkyl” group is-(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O) OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18th eds., Mack Publishing, Easton PA (1990) or Remington: The Science and Practice of Pharmacy, 19th eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers.

Throughout this specification and the Aspects which follow, the term “Cn-m” indicates a range which includes the endpoints, wherein n and m are integers and indicate the number of carbons. Examples include C₁₋₈, and C₁₋₆.

To optimize the synthesis of Sonrotoclax and to yield a more stable intermediate, pharmaceutically accepted alcohols including methanol, ethanol, isopropanol, glycol and 1,3-propandiol, were used for ketone protection of 1-azaspiro[3.5]nonan-2-one. However, solids with good physical form and high purity were obtained only when the 1,3-propandiol was used.

Considering the poor mobility of 1,3-propandiol, the reaction solvents were screened. After considering alcohols, esters, and ethers, IPAc was chosen as reaction solvent as well as the solvent dissolving hydrogen chloride, owing to its acceptable stability in hydrogen chloride condition. Triethyl orthoformate was selected as dehydration reagent to replace trimethyl orthoformate, to avoid unfavorable reactions.

This selection of alcohol, reaction solvent, and dehydration reagent results in solid products with a purity of 99.9%. In addition, the product is highly stable with no increase of impurities when stored under 2-8° C. for 3.5 months.

In a first aspect, disclosed herein a compound of Formula (I),

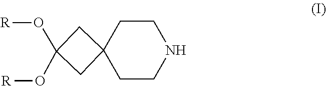

or a salt thereof, or a stereoisomer thereof,
wherein
at each of its occurrences, R is each independently C₂₋₆alkyl or C₃₋₆cycloalkyl, each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl.

In one embodiment of the first aspect, at each of its occurrences, R is each independently ethyl, propyl, butyl, pentyl, hexyl, cyclopropyl, cyclobutyl, cyclopentyl, or cyclohexyl; each of said ethyl, propyl, butyl, pentyl, hexyl, cyclopropyl, cyclobutyl, cyclopentyl, or cyclohexyl is optionally substituted with at least one substituent selected from F, Cl, Br, I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and R^{1c} are each independently selected from hydrogen, methyl, ethyl, propyl, butyl, pentyl, hexyl, cyclopropyl, cyclobutyl, cyclopentyl, cyclohexyl, -haloC₁₋₆alkyl or -haloC₃₋₆cycloalkyl;
preferably, R is each independently ethyl, propyl, butyl, pentyl, hexyl, cyclopropyl, cyclobutyl, cyclopentyl, or cyclohexyl; each of said ethyl, propyl, butyl, pentyl, hexyl, cyclopropyl, cyclobutyl, cyclopentyl, or cyclohexyl is optionally substituted with at least one substituent selected from F, Cl, Br or I;
more preferably, R is each independently ethyl, propyl, butyl, pentyl, hexyl, cyclopropyl, cyclobutyl, cyclopentyl, or cyclohexyl;
even more preferably, R is each independently ethyl, propyl (n-propyl or iso-propyl) or butyl (n-butyl, sec-butyl, iso-butyl, tert-butyl).

In another embodiment of the first aspect, two R together with the two oxygen atoms to which each is attached, form a 5-, 6-, 7-, 8-, 9-, 10-, 11- or 12-membered ring, said ring is optionally substituted with at least one substituent selected from F, Cl, Br, I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};

at each of its occurrences, R^{1a}, R^{1b} and R^{1c} are each independently selected from hydrogen, methyl, ethyl, propyl, butyl, pentyl, hexyl, cyclopropyl, cyclobutyl, cyclopentyl, cyclohexyl, -haloC₁₋₆alkyl or -haloC₃₋₆cycloalkyl;

preferably, two R together with the two oxygen atoms to which each is attached, form a 5-, 6-, 7- or 8-membered ring, said ring is optionally substituted with at least one substituent selected from F, Cl, Br, I, methyl, ethyl, propyl, butyl, pentyl, hexyl, heptyl, octyl, cyclopropyl, cyclobutyl, cyclopentyl, cyclohexyl, cycloheptyl or cyclooctyl;

more preferably, two R together with the two oxygen atoms to which each is attached, form a 5-, 6-, 7- or 8-membered ring, said ring is optionally substituted with at least one substituent selected from F, Cl, Br, I;

even more preferably, two R together with the two oxygen atoms to which each is attached, form a 5-, 6-, 7- or 8-membered ring.

In a preferred embodiment of the above aspect, the compound is selected from the group consisting of:

| 1 | 2 | 3 | 4 |
|---|---|---|---|
| 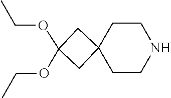 | 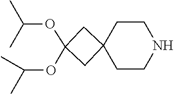 [SMILES: CC(C)OC1(OC(C)C)CC2(CCNCC2)C1] | 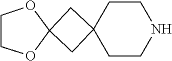 [SMILES: C1CC2(CCN1)CC1(C2)OCCO1] | 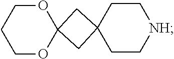 [SMILES: C1COC2(CC3(CCNCC3)C2)OC1] |

preferably the compound is

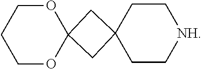 [SMILES: C1COC2(CC3(CCNCC3)C2)OC1]

When

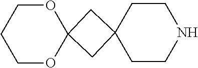 [SMILES: C1COC2(CC3(CCNCC3)C2)OC1]

is used as an intermediate in the process for preparing Sonrotoclax, especially for preparation of the intermediate methyl 2-((1H-pyrrolo[2,3-b]pyridin-5-yl)oxy)-4-(1,5-dioxa-11-azadispiro[5.1.5⁸.1⁶]tetradecan-11-yl)benzoate, it can greatly increase the yield and purity.

### Method of Making

In a second aspect, disclosed herein a method for preparing the compound of Formula (I), comprising Step (A): reacting a compound of Formula (II) with dehydration reagent and a reactant alcohol in the presence of acid and a solvent to obtain the product of Formula (I)

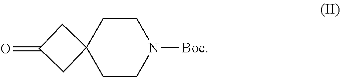 [SMILES: CC(C)(C)OC(=O)N1CCC2(CC1)CC(=O)C2]

In one embodiment of the second aspect, the reaction temperature of Step (A) is about 10-80° C.; preferably the temperature of the method is about 15-60° C.; more preferably the temperature of the method is about 20-40° C.; even more preferably the temperature of the method is about 20-30° C.

In another embodiment of the second aspect, the reaction time of Step (A) is about 10-20 hours; preferably, the reaction time of the method is about 12-18 hours; more preferably, the reaction time of the method is about 16 hours.

In another embodiment of the second aspect, the reactant alcohol is selected from the group consisting of methanol, ethanol, isopropanol, glycol, ethylene glycol, 1,3-propylene glycol, 2,2-dimethylcyclopentanol, and cyclohexanol; preferably is selected from ethanol, isopropanol, glycol, ethylene glycol, and 1,3-propylene glycol; and more preferably is 1,3-propylene glycol.

In another embodiment of the second aspect, the reactant alcohol in the reaction ranges from about 1 molar equivalent to about 20 molar equivalents relative to the amount of the compound of Formula (II); preferably the reactant alcohol in the reaction ranges from about 1 molar equivalent to about 10 molar equivalents relative to the amount of the compound of Formula (II); more preferably the reactant alcohol in the reaction ranges from about 2 molar equivalent to about 8 molar equivalents relative to the amount of the compound of Formula (II); even more preferably the reactant alcohol in the reaction ranges from about 2 molar equivalent to about 4 molar equivalents relative to the amount of the compound of Formula (II); even more preferably the reactant alcohol in the reaction ranges from about 3 molar equivalent relative to the amount of the compound of Formula (II).

In another embodiment of the second aspect, the dehydration reagent is selected from the group consisting of aluminum oxide, anhydrous zinc chloride, concentrated sulfuric acid, phosphoric acid, triethyl orthoformate, sodium borohydride, and trimethyl orthoformate; preferably is selected from triethyl orthoformate and trimethyl orthoformate; and more preferably is triethyl orthoformate.

In another embodiment of the second aspect, the dehydration reagent in the reaction ranges from about 0.5 molar equivalent to about 4 molar equivalents relative to the amount of the compound of Formula (II); preferably ranges from about 0.9 to 3; more preferably about 1.3.

In another embodiment of the second aspect, the acid is selected from the group consisting of acetic acid, hydrochloric acid, formic acid, and ascorbic acid; preferably is selected from hydrochloric acid and formic acid; and more preferably is hydrochloric acid.

In another embodiment of the second aspect, the acid ranges from about 1 molar equivalent to about 5 molar equivalents relative to the amount of the compound of Formula (II); preferably ranges from about 2 to 4, more preferably about 3.5.

In another embodiment of the second aspect, the solvent is selected from the group consisting of isopropyl acetate, ethyl acetate, hexane, heptane, dichloromethane, tetrahydrofuran, acetonitrile, dimethylformamide, toluene, dimethyl sulfoxide, and any mixture thereof; preferably is selected from isopropyl acetate.

In another embodiment of the second aspect, the method further comprises
Step (B): the mixture is added to non to low polarity solvent to obtain the solid compound of Formula (I).

In a preferred embodiment of the second aspect, the said non to low polarity solvent is selected from Methyl Tertiary Butyl Ether, Et₂O, hexane, cyclohexane, i-propyl ether, toluene, xylene, tetrahydrofuran, dioxane, or mixture thereof; preferably, the said non to low polarity solvent is selected from Methyl Tertiary Butyl Ether.

In a preferred embodiment of the second aspect, the mixture is aged for about 1-10 hours after adding non to low polarity solvent, preferably the mixture is aged for about 3-7 hrs; more preferably the mixture is aged for about 6 hrs.

In a preferred embodiment of the second aspect, the wet cake of solid compound of Formula (I) is collected from centrifugation or filtration, and the wet cake is dried under vacuum at about 10-60° C., preferably at about 15-55° C., more preferably at about 35-45° C.

In a preferred embodiment of the second aspect, the wet cake is dried for 10-40 hours, preferably for 20-30 hours, more preferably for 20 hours.

### Method of Making Sonrotoclax

In a third aspect, disclosed herein is a method of the second aspect, wherein the compound of Formula (I) is

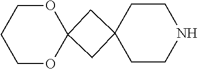 [SMILES: C1COC2(CC3(CCNCC3)C2)OC1]

or the salt thereof.

Provided here is a method of making Sonrotoclax. In one embodiment, the method comprises reacting the compound of Formula (I) with a compound of Formula (SI):

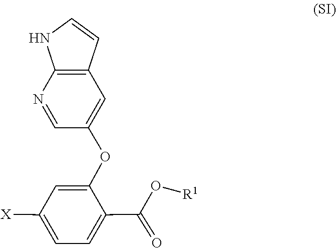

in the presence of a base,
wherein X is halogen; preferably, X is F, Cl, Br, or I; more preferably, X is F; and
R¹ is C₁₋₆alkyl, or C₃₋₆cycloalkyl, wherein each of said C₁₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl,
to provide a compound of Formula (SII),

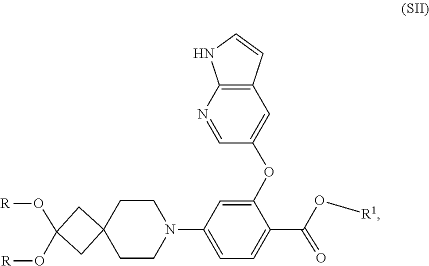

or a salt thereof.

In one embodiment, the base is 1,8-Diazabicyclo(5.4.0) undec-7-ene (DBU).

In one embodiment, the reaction happens between about 65° C. and about 75° C., at about 65° C., about 70° C., or about 75° C.

Provided here is a method of making Sonrotoclax. In one embodiment, the method comprises reacting the compound of Formula (SII) with an acid or a base to provide a compound of Formula (SIII),

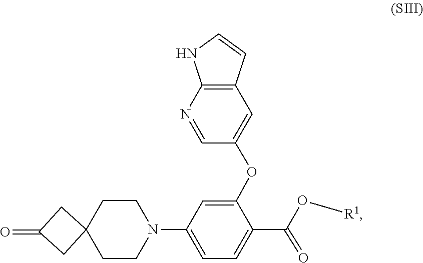

or a salt thereof.

In one embodiment, the acid is HCl, or the base is NaOH.

Provided here is a method of making Sonrotoclax. In one embodiment, the method comprises reacting the compound of Formula (SIII) with (S)-2-(2-isopropylphenyl) pyrrolidine, or a salt thereof, to provide a compound of Formula (SIV)

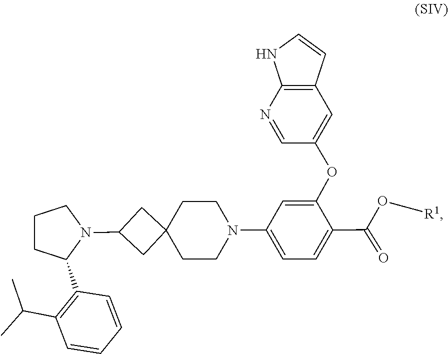

or a salt thereof.

In one embodiment, the reaction happens in the presence of NaBH(OAc)₃.

In one embodiment, the reaction happens at less than about 30° C.

Provided here is a method of making Sonrotoclax. In one embodiment, the method comprises reacting the compound of Formula (SIV), or a salt thereof, with 4-((((1r,4r)-4-hydroxy-4-methylcyclohexyl)methyl)amino)-3-nitrobenzenesulfonamide, to provide Sonrotoclax,

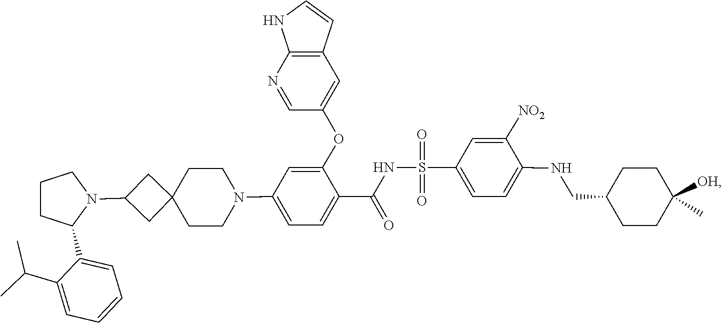 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof.

In one embodiment, the reaction happens in the presence of 1-ethyl-3-(3-dimethylaminopropyl)carbodiimide (EDCI) and 4-dimethylaminopyridine (DMAP).

Provided here is a method of making Sonrotoclax. In one embodiment, the method comprises reacting the compound of Formula (SIV), or a salt thereof, with an acid or a base, to provide (S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid,

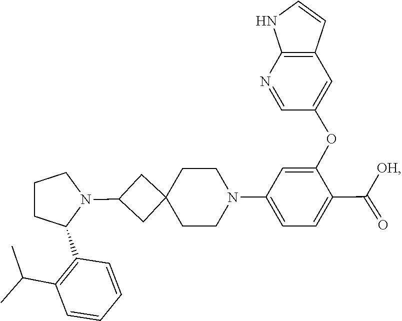 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof.

In one embodiment, the acid is HCl, or the base is NaOH.

Provided here is a method of making Sonrotoclax. In one embodiment, the method comprises reacting(S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid,

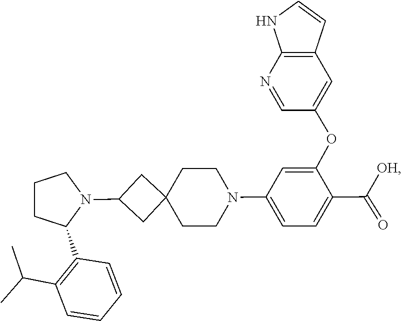 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof, with 4-((((1r,4r)-4-hydroxy-4-methylcyclohexyl)methyl)amino)-3-nitrobenzenesulfonamide, to provide Sonrotoclax,

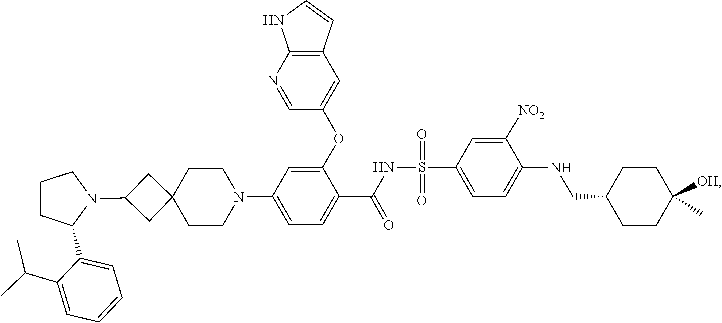 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof.

In one embodiment, the reaction happens in the presence of EDCI and DMAP.

In one embodiment, provided here is a method for preparing a pharmaceutical composition comprising Sonrotoclax, comprising mixing Sonrotoclax with a pharmaceutically acceptable excipient, wherein Sonrotoclax is prepared according to the method provided here.

### NUMBERED EMBODIMENTS

Embodiment 1. A compound of Formula (I),

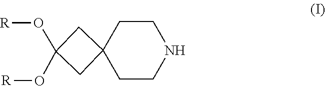

or a salt thereof, or a stereoisomer thereof,
wherein
at each of its occurrences, R is each independently C₂₋₆alkyl or C₃₋₆cycloalkyl, wherein each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and Ric are each independently selected from hydrogen, C₁₋₆alkyl, C₃₋₆cycloalkyl, -haloC₁₋₆alkyl or -haloC₃₋₆cycloalkyl.

Embodiment 2. The compound of embodiment 1, wherein at each of its occurrences, R is each independently ethyl, propyl, butyl, pentyl, hexyl, cyclopropyl, cyclobutyl, cyclopentyl, or cyclohexyl; each of said ethyl, propyl, butyl, pentyl, hexyl, cyclopropyl, cyclobutyl, cyclopentyl, or cyclohexyl is optionally substituted with at least one substituent selected from F, Cl, Br, I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, Ra, R^{1b} and R^{1c} are each independently selected from hydrogen, methyl, ethyl, propyl, butyl, pentyl, hexyl, cyclopropyl, cyclobutyl, cyclopentyl, cyclohexyl, -haloC₁₋₆alkyl or -haloC₃₋₆cycloalkyl;
preferably, R is each independently ethyl, propyl, butyl, pentyl, hexyl, cyclopropyl, cyclobutyl, cyclopentyl, or cyclohexyl; each of said ethyl, propyl, butyl, pentyl, hexyl, cyclopropyl, cyclobutyl, cyclopentyl, or cyclohexyl is optionally substituted with at least one substituent selected from F, Cl, Br or I;
more preferably, R is each independently ethyl, propyl, butyl, pentyl, hexyl, cyclopropyl, cyclobutyl, cyclopentyl, or cyclohexyl;
even more preferably, R is each independently ethyl, propyl (n-propyl or iso-propyl) or butyl (n-butyl, sec-butyl, iso-butyl, tert-butyl).

Embodiment 3. The compound of embodiment 1, wherein two R together with the two oxygen atoms to which each is attached, form a 5-, 6-, 7-, 8-, 9-, 10-, 11- or 12-membered ring, said ring is optionally substituted with at least one substituent selected from F, Cl, Br, I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and R^{1c} are each independently selected from hydrogen, methyl, ethyl, propyl, butyl, pentyl, hexyl, cyclopropyl, cyclobutyl, cyclopentyl, cyclohexyl, -haloC₁₋₆alkyl or -haloC₃₋₆cycloalkyl;
preferably, two R together with the two oxygen atoms to which each is attached, form a 5-, 6-, 7- or 8-membered ring, said ring is optionally substituted with at least one substituent selected from F, Cl, Br, I, methyl, ethyl, propyl, butyl, pentyl, hexyl, heptyl, octyl, cyclopropyl, cyclobutyl, cyclopentyl, cyclohexyl, cycloheptyl or cyclooctyl;
more preferably, two R together with the two oxygen atoms to which each is attached, form a 5-, 6-, 7- or 8-membered ring, said ring is optionally substituted with at least one substituent selected from F, Cl, Br, I;
even more preferably, two R together with the two oxygen atoms to which each is attached, form a 5-, 6-, 7- or 8-membered ring.

Embodiment 4. The compound of any of embodiments 1-3, wherein the compound is selected from the group consisting of:

| 1 | 2 | 3 | 4 |
|---|---|---|---|
| 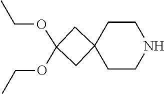 | 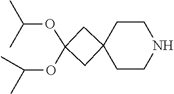 [SMILES: CC(C)OC1(OC(C)C)CC2(CCNCC2)C1] | 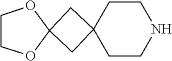 [SMILES: C1CC2(CCN1)CC1(C2)OCCO1] | 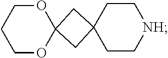 [SMILES: C1COC2(CC3(CCNCC3)C2)OC1] |

preferably the compound is

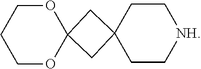 [SMILES: C1COC2(CC3(CCNCC3)C2)OC1]

Embodiment 5. A method for preparing the compound of Formula (I) of embodiment 1, comprising Step (A): reacting a compound of Formula (II) with dehydration reagent and a reactant alcohol in the presence of acid and a solvent to obtain the product of Formula (I)

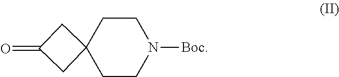 [SMILES: CC(C)(C)OC(=O)N1CCC2(CC1)CC(=O)C2]

Embodiment 6. The method of embodiment 5, wherein, the reaction temperature of Step (A) is about 10-80° C.; preferably the temperature of the method is about 15-60° C.; more preferably the temperature of the method is about 20-40° C.; even more preferably the temperature of the method is about 20-30° C.

Embodiment 7. The method of embodiment 5, wherein, the reaction time of Step (A) is about 10-20 hours; preferably, the reaction time of the method is about 12-18 hours; more preferably, the reaction time of the method is about 16 hours.

Embodiment 8. The method of embodiment 5, wherein the reactant alcohol is selected from the group consisting of methanol, ethanol, isopropanol, glycol, ethylene glycol, 1,3-propylene glycol, 2,2-dimethylcyclopentanol, and cyclohexanol; preferably is selected from ethanol, isopropanol, glycol, ethylene glycol, and 1,3-propylene glycol; and more preferably is 1,3-propylene glycol.

Embodiment 9. The method of embodiment 5, wherein the reactant alcohol in the reaction ranges from about 1 molar equivalent to about 20 molar equivalents relative to the amount of the compound of Formula (II); preferably the reactant alcohol in the reaction ranges from about 1 molar equivalent to about 10 molar equivalents relative to the amount of the compound of Formula (II); more preferably the reactant alcohol in the reaction ranges from about 2 molar equivalent to about 8 molar equivalents relative to the amount of the compound of Formula (II); even more preferably the reactant alcohol in the reaction ranges from about 2 molar equivalent to about 4 molar equivalents relative to the amount of the compound of Formula (II); even more preferably the reactant alcohol in the reaction ranges from about 3 molar equivalent relative to the amount of the compound of Formula (II).

Embodiment 10. The method of embodiment 5, wherein the dehydration reagent is selected from the group consisting of aluminum oxide, anhydrous zinc chloride, concentrated sulfuric acid, phosphoric acid, triethyl orthoformate, sodium borohydride, and trimethyl orthoformate; preferably is selected from triethyl orthoformate and trimethyl orthoformate; and more preferably is triethyl orthoformate.

Embodiment 11. The method of embodiment 5, wherein the dehydration reagent in the reaction ranges from about 0.5 molar equivalent to about 4 molar equivalents relative to the amount of the compound of Formula (II); preferably ranges from about 0.9 to 3; more preferably about 1.3.

Embodiment 12. The method of embodiment 5, wherein the acid is selected from the group consisting of acetic acid, hydrochloric acid, formic acid, and ascorbic acid; preferably is selected from hydrochloric acid and formic acid; and more preferably is hydrochloric acid.

Embodiment 13. The method of embodiment 5, wherein the acid ranges from about 1 molar equivalent to about 5 molar equivalents relative to the amount of the compound of Formula (II); preferably ranges from about 2 to 4, more preferably about 3.5.

Embodiment 14. The method of embodiment 5, wherein the solvent is selected from the group consisting of isopropyl acetate, ethyl acetate, hexane, heptane, dichloromethane, tetrahydrofuran, acetonitrile, dimethylformamide, toluene, dimethyl sulfoxide, and any mixture thereof; preferably is selected from isopropyl acetate.

Embodiment 15. The method of embodiment 5, further comprising
Step (B): the mixture is added non to low polarity solvent to obtain the solid compound of Formula (I).

Embodiment 16. The method of embodiment 15, wherein the said non to low polarity solvent is selected from Methyl Tertiary Butyl Ether, Et₂O, hexane, cyclohexane, i-propyl ether, toluene, xylene, tetrahydrofuran, dioxane, or mixture thereof; preferably, the said non to low polarity solvent is selected from Methyl Tertiary Butyl Ether.

Embodiment 17. The method of embodiment 15, wherein the mixture is aged for about 1-10 hours after adding non to low polarity solvent, preferably the mixture is aged for about 3-7 hrs; more preferably the mixture is aged for about 6 hrs.

Embodiment 18. The method of embodiment 15, wherein the wet cake of solid compound of Formula (I) is collected from centrifugation or filtration, and the wet cake is dried under vacuum at about 10-60° C., preferably at about 15-55° C., more preferably at about 35-45° C.

Embodiment 19. The method of embodiment 18, wherein the wet cake is dried for 10-40 hours, preferably for 20-30 hours, more preferably for 20 hours.

Embodiment 20. The method of any one of embodiments 5-19, wherein the compound of Formula (I) is

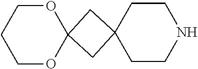 [SMILES: C1COC2(CC3(CCNCC3)C2)OC1]

or the salt thereof.

### EXAMPLES

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature to freezing temperature. A given reaction can be carried out in one solvent or mixture of solvents.

The selection of an appropriate protecting group can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

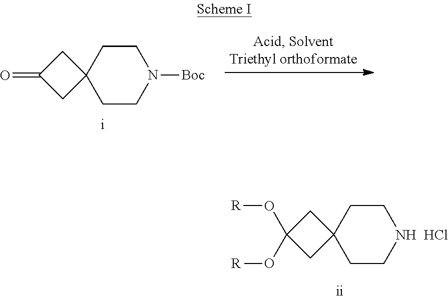

Compounds of Formula (I) can be prepared as shown in Scheme I. The ketal reaction, which is aided with acid and triethyl orthoformate, is between an alcohol and compound (I) to afford compound (II).

A nitrogen inerted reactor is charged with compound (I) followed by solvents and set to stir. The vessel is charged with triethyl orthoformate, followed by the addition of an alcohol. The vessel is fed with acid while keeping the batch temperature at 25±5° C. for 10-20 hours to obtain compound (II).

### Workup

Upon reaction completion, the batch is charged with Methyl Tertiary Butyl Ether while keeping the batch temperature at 25±5° C. The mixture is aged for approximately 6 hrs and subsequently centrifuged to collect the wet cake. The cake is washed with Methyl Tertiary Butyl Ether.

### Drying

The wet cake is dried under vacuum at 40±5° C. for 20 h. Drying is considered completed when Isopropyl Acetate is ≤5000 ppm; Methyl Tertiary Butyl Ether is ≤5000 ppm; and Water content ≤0.5%.

### Abbreviations

EtOH Ethanol
IPAc Isopropyl acetate
TEOF Triethyl orthoformate
HCl Hydrogen chloride
MTBE Methyl Tertiary Butyl Ether
LDPE Low Density Polyethylene
NMR Nuclear Magnetic Resonance Spectroscopy
UV Ultraviolet
HPLC High Performance Liquid Chromatography
LC-MS Liquid Chromatograph Mass Spectrometer
TLC Thin Layer Chromatography
GC Gas chromatography

### Example 1:2,2-diethoxy-7-azaspiro[3.5]nonane hydrogen chloride

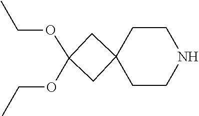

Tert-butyl 2-oxo-7-azaspiro[3.5]nonane-7-carboxylate (10.0 g, 41.8 mmol) was charged in a nitrogen inert reactor. To the reactor was added EtOH (20.0 ml) to obtain a mixture. The mixture was stirred at 20-30° C. for 1 h. To this mixture was added triethyl orthoformate (8.1 g, 54.7 mmol) and then 15% HCl/EtOH solution (36.0 g, 146.3 mmol) while the temperature was kept at 20-30° C. for 20 hrs to yield the product. The product was tested by GC (carrier gas: helium (He)). Purity: 74.3%. GC-MS: 213.2.

### Example 2:2,2-diisopropoxy-7-azaspiro[3.5]nonane hydrogen chloride

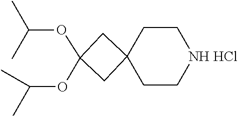

Tert-butyl 2-oxo-7-azaspiro[3.5]nonane-7-carboxylate (10.0 g, 41.8 mmol) was charged in a nitrogen inerted reactor. To the reactor was added isopropanol (20.0 ml) to obtain a mixture. The mixture was stirred at 20-30° C. for 1 h. To this mixture was added triethyl orthoformate (8.1 g, 54.7 mmol) and then 15% HCl/isopropanol solution (36.0 g, 146.3 mmol) while keeping the batch temperature at 20-30° C. for approximately 20 hrs to yield the product. The product was tested by GC (carrier gas: helium (He)). Only 5% of 2,2-diisopropoxy-7-azaspiro[3.5]nonane hydrogen chloride was obtained.

### Example 3:1,4-dioxa-10-azadispiro[4.1.5.1]tridecane hydrogen chloride

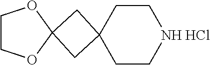

Tert-butyl 2-oxo-7-azaspiro[3.5]nonane-7-carboxylate (10.0 g, 41.8 mmol) was charged in a nitrogen inerted reactor. To the reactor was added IPAc (20.0 ml) to obtain a mixture. The mixture was stirred at 20-30° C. for 1 h. To this mixture was added triethyl orthoformate (8.1 g, 54.7 mmol) and then ethylene glycol (8.2 g, 132.1 mmol). The reactor was fed with 15% HCl/IPAc solution (36.0 g, 146.3 mmol), while keeping the batch temperature at 20-30° C. for approximately 20 hrs to yield the product. The product was tested by GC (carrier gas: helium (He)) which shows that 99.9% of 1,4-dioxa-10-azadispiro[4.1.5.1]tridecane hydrogen chloride was obtained. However, no solid was separated out. GC-MS: 183.1

### Example 4:1,5-dioxa-11-azadispiro[5.1.5.1]tetradecane hydrogen chloride

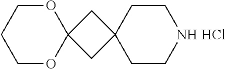

Tert-butyl 2-oxo-7-azaspiro[3.5]nonane-7-carboxylate (10.0 g, 41.8 mmol) was charged in a nitrogen inerted reactor. To the reactor was added IPAc (20.0 ml) to obtain a mixture. The mixture was stirred at 20-30° C. for 1 h. To this mixture was added triethyl orthoformate (8.1 g, 54.7 mmol) and then 1,3-propylene glycol (10.0 g, 129.6 mmol). The reactor was fed with 15% HCl/IPAc solution (36.0 g, 146.3 mmol), while keeping the batch temperature at 20-30° C. for approximately 20 hrs. The product was tested by GC (carrier gas: helium (He)) which shows that 99.9% of 1,5-dioxa-11-azadispiro[5.1.5.1]tetradecane hydrogen chloride was obtained and the solid was well separated out. GC-MS: 197.1

### Example 5:1,5-dioxa-11-azadispiro[5.1.5.1]tetradecane hydrogen chloride

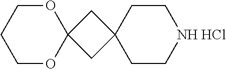

Tert-butyl 2-oxo-7-azaspiro[3.5]nonane-7-carboxylate (10.0 g, 41.8 mmol) was charged in a nitrogen inerted reactor. To the reactor was added IPAc (20.0 ml) to obtain a mixture. The mixture was stirred at 20-30° C. for 1 h. To this mixture was added triethyl orthoformate (8.1 g, 54.7 mmol) and then 1,3-propylene glycol (10.0 g, 129.6 mmol). The reactor was fed with of 15% HCl/IPAc solution (36.0 g, 146.3 mmol) while keeping the batch temperature at 20-30° C. for approximately 16 hours. Upon reaction completion, the batch is charged with Methyl Tertiary Butyl Ether (MTBE, 27 ml) while keeping the batch temperature at 25±5° C. The mixture is aged for approximately 6 hrs and subsequently centrifuged to collect the wet cake. The cake is washed with MTBE (27 ml). The wet cake is dried under vacuum at 40±5° C. for 20 h to obtain solid product 1,5-dioxa-11-azadispiro[5.1.5.1]tetradecane hydrogen chloride. ¹H NMR (400 MHz, d-DMSO) δ 8.908 (s, 2H), 3.722 (t, 4H), 2.924 (m, 4H), 1.906 (s, 4H), 1.692 (t, 4H), 1.554 (t, 2H). GC-MS: 197.1

### Example 6: Stability Test of 1,5-dioxa-11-azadispiro[5.1.5.1]tetradecane hydrogen chloride

1,5-dioxa-11-azadispiro[5.1.5.1]tetradecane hydrogen chloride (2.0 g, 8.4 mmol) was stored in 2˜8° C. and the packaged in double layered LDPE bag with desiccant between the two layers, inside a heat-sealed aluminum foil bag for 3.5 months. The sample was tested by GC (carrier gas: N2) and HPLC (Column: Waters Xselect HSS T3 (150 mm*4.6 mm, 3.5 um) P/N: 186004786; mobile phase 0.05% TFA in water, ACN:MeOH=1:1, v/v; Flow: 1 ml/min; Temperature: 30° C.). The result in Table 1 shows that intermediate 1,5-dioxa-11-azadispiro[5.1.5.1]tetradecane hydrogen chloride was stable enough in this condition.

TABLE 1

| | Product, Impurity profile (RRT, Area %) | | |
|---|---|---|---|
| Storage | 1.00 | | |
| condition | | | |
| | 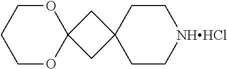 | 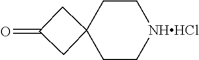 | 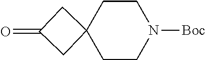 [SMILES: CC(C)(C)OC(=O)N1CCC2(CC1)CC(=O)C2] |
| 2-8° C. | 99.90 | | |

for 3.5
Months

### Example 7: methyl 2-((1H-pyrrolo[2,3-b]pyridin-5-yl)oxy)-4-(1,5-dioxa-11-azadispiro[5.1.5⁸.1⁶]tetradecan-11-yl)benzoate

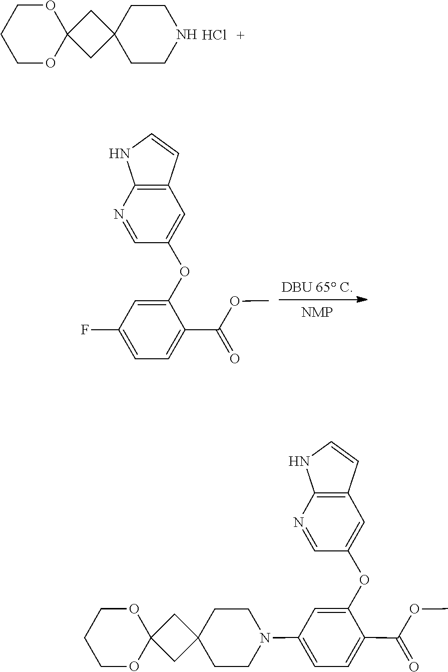 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

Methyl 2-((1H-pyrrolo[2,3-b]pyridin-5-yl)oxy)-4-fluorobenzoate was synthesized per the methods in WO2019210828 (e.g., Example B12), which is incorporated by reference in its entirety. To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 65° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.62%, yield: 83%. ¹H NMR (400 MHZ, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 8: methyl 2-((1H-pyrrolo[2,3-b]pyridin-5-yl)oxy)-4-(1,5-dioxa-11-azadispiro[5.1.5⁸.1⁶]tetradecan-11-yl)benzoate

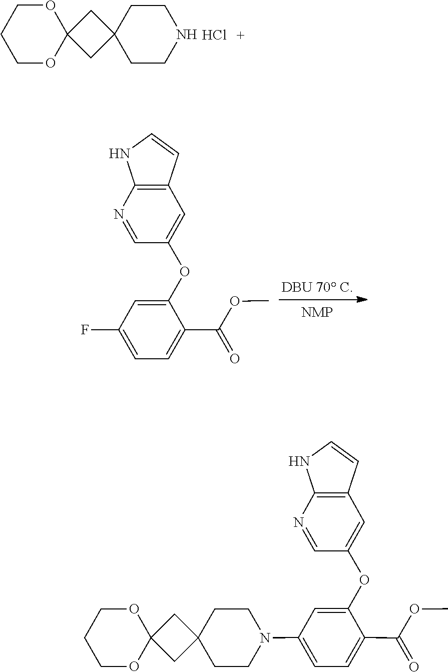 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 70° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.26%, yield: 83%. ¹H NMR (400 MHZ, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 9: methyl 2-((1H-pyrrolo[2,3-b]pyridin-5-yl)oxy)-4-(1,5-dioxa-11-azadispiro[5.1.5⁸.1⁶]tetradecan-11-yl)benzoate (Compound 1)

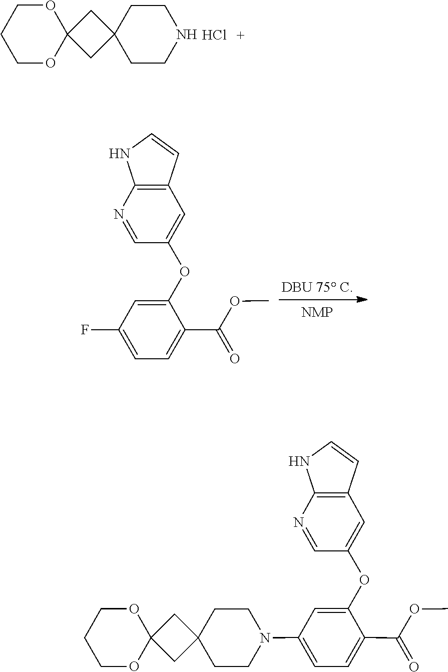 [SMILES: C1COC2(CC3(CCNCC3)C2)OC1 ; Cl ; COC(=O)c1ccc(F)cc1Oc1cnc2[nH]ccc2c1 ; COC(=O)c1ccc(N2CCC3(CC2)CC2(C3)OCCCO2)cc1Oc1cnc2[nH]ccc2c1]

To a suspension of methyl 2-((1H-pyrrolo[2,3-b]pyridin-5-yl)oxy)-4-fluorobenzoate (1.00 g, 3.49 mmol) and 1,5-dioxa-11-azadispiro[5.1.5⁸.1⁶]tetradecane hydrochloride (1.30 g, 5.58 mmol) in NMP (5 mL), followed by the addition of DBU (1.50 g, 9.85 mmol). The resulting solution was stirred for 24 h at 75° C., cooled to 50° C. The pH value adjustment was carried out to the target value 7 with AcOH, and then water (1.5 mL) was charged and followed by seed (0.01 g). The mixture solution was stirred at 50° C. for 6 h and then cooled to 20° C. The wet cake was collected by filtering and washing with water. The obtained wet cake was slurried with the mixture solvents 2-MeTHF (8 mL) and n-Heptane (2 mL). The product was obtained after drying. Purity: 95.25%, yield: 81%. ¹H NMR (400 MHZ, DMSO-d6) δ 11.63 (s, 1H), 8.0 (d, 1H), 7.76 (d, 1H), 7.48 (t, 1H), 7.42 (d, 1H), 6.79 (dd, 1H), 6.38-6.40 (m, 2H), 3.72 (t, 4H), 3.65 (s, 3H), 3.16 (t, 4H), 1.95 (s, 1H), 1.52-1.57 (m, 6H).

### Example 10:2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (Sonrotoclax)

### Step 1: methyl 2-((1H-pyrrolo[2,3-b]pyridin-5-yl)oxy)-4-(2-oxo-7-azaspiro[3.5]nonan-7-yl)benzoate

To the solution of methyl 2-((1H-pyrrolo[2,3-b]pyridin-5-yl)oxy)-4-(1,5-dioxa-11-azadispiro[5.1.5⁸.1⁶]tetradecan-11-yl)benzoate (Compound 1) (180 g, 0.39 mol) in DCM (2 L) was added diluted HCl acid (1M, 1.5 L) and stirred for overnight. After the reaction was completed, the mixture was cooled to 10° C. and was adjusted to pH=8-9 with aqueous NaOH solution (4 M) under stirring. The organic phase was separated and washed with 15% aq. NaCl (1 L), then washed with H₂O (1 L). After the organic phase was concentrated to 500 mL, MTBE (1 L) was poured into the solution and then the system was concentrated to 500 mL (repeated this work-up 3 times). The resulting system was stirred for 0.5 hour. After filtration, the cake was collected and then dried in vacuum to obtain the tittle product as a white solid (152 g, yield: 96%). ¹H NMR (400 MHZ, DMSO-d6) δ ppm: 11.64 (s, 1H), 8.02 (d, J=2.4 Hz, 1H), 7.78 (d, J=9.2 Hz, 1H), 7.47 (t, J=3.2 Hz, 1H), 7.44 (d, J=2.4 Hz, 1H), 6.83 (dd, J=2.4 Hz, J=9.2 Hz, 1H), 6.43 (d, J=2.4 Hz, 1H), 6.38-6.36 (m, 1H), 3.65 (s, 3H), 3.24-3.21 (m, 4H), 2.80 (s, 4H), 1.70-1.67 (m, 4H). MS (ESI, m/e) [M+1]⁺ 405.9.

### Step 2: (S)-tert-butyl 2-(2-(prop-1-en-2-yl)phenyl) pyrrolidine-1-carboxylate

To a mixture of (S)-tert-butyl 2-(2-bromophenyl) pyrrolidine-1-carboxylate (50 g, 153.3 mmol) and 4,4,5,5-tetramethyl-2-(prop-1-en-2-yl)-1,3,2-dioxaborolane (38.6 g, 229.9 mmol) in dioxane (500 mL) and H₂O (50 mL) was added Cs₂CO₃ (100 g, 305 mmol) and Pd(dppf)Cl₂ (6.6 g, 7.5 mmol). The mixture was stirred at 100° C. for 8 hours. TLC showed the reaction was completed. The mixture was concentrated in vacuum. The residue was purified by column chromatography on silica gel (eluent: PE/EA (v/v)=100/1 to 10/1) to obtain(S)-tert-butyl 2-(2-(prop-1-en-2-yl)phenyl) pyrrolidine-1-carboxylate (65 g, crude). The crude product was used directly in next step.

### Step 3: (S)-tert-butyl 2-(2-isopropylphenyl) pyrrolidine-1-carboxylate

To a solution of (S)-tert-butyl 2-(2-(prop-1-en-2-yl)phenyl) pyrrolidine-1-carboxylate (30 g, 104.39 mmol) in MeOH (500 mL) was added Pd/C (10 g, 10%) and the mixture was stirred at 20° C. under H₂ (15 Psi) for 12 hours. TLC showed the reaction was completed. The mixture was filtered and the filtrate was concentrated in vacuum to give(S)-tert-butyl 2-(2-isopropylphenyl) pyrrolidine-1-carboxylate (60 g, crude), which was used in next step without further purification. ¹H NMR (400 MHZ, CDCl₃) δ ppm: 7.39-6.90 (m, 4H), 5.36-5.04 (m, 1H), 3.77-3.52 (m, 2H), 3.20-3.17 (m, 1H), 2.47-2.24 (m, 1H), 1.96-1.65 (m, 3H), 1.54-1.38 (m, 2H), 1.31-1.22 (m, 8H), 1.17 (s, 7H).

### Step 4: (S)-2-(2-isopropylphenyl) pyrrolidine hydrochloride

To a solution of tert-butyl 2-(2-isopropylphenyl) pyrrolidine-1-carboxylate (55 g, 190 mmol) in DCM (50 mL) was added HCl in 1,4-dioxane (4 M, 142 mL, 570 mmol) dropwise at room temperature. The mixture was stirred at room temperature for overnight. The mixture was concentrated in vacuum. The resulting residue was slurried with EA (100 mL) and then filtered, dried in vacuum to give (S)-2-(2-isopropylphenyl) pyrrolidine hydrochloride 26 g (yield: 60.4%). ¹H NMR (400 MHZ, DMSO-d6) δ ppm: 9.93 (s, 1H), 8.81 (s, 1H), 7.63-7.57 (m, 1H), 7.41-7.34 (m, 2H), 7.32-7.24 (m, 1H), 4.91-4.75 (m, 1H), 3.47-3.35 (m, 1H), 3.31-3.25 (m, 1H), 2.40-2.21 (m, 1H), 2.19-1.86 (m, 3H), 1.25 (d, J=6.7 Hz, 3H), 1.17 (d, J=6.7 Hz, 3H). MS (ESI, m/e) [M+1]⁺ 190.0.

### Step 5: methyl(S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoate

A mixture of (S)-2-(2-isopropylphenyl) pyrrolidine hydrochloride (120 g, 0.535 mole) and methyl 2-((1H-pyrrolo[2,3-b]pyridin-5-yl)oxy)-4-(2-oxo-7-azaspiro[3.5]nonan-7-yl)benzoate (218 g, 0.509 mole) in DCM (2.2 L) was charged into a reactor. The temperature was controlled below 30° C. and NaBH(OAc)₃ (216 g, 1.018 mole) was added into the reactor in 5-6 portions. Then the reaction mixture was stirred at room temperature and monitored by TLC. After the starting material ketone was consumed completely, the mixture was adjusted to pH=4˜5 with diluted HCl acid (0.5 M). The separated organic phase was washed with H₂O (600 mL×2) and then washed with aq. NaHCO₃ (600 mL×2), saturated aq. NaCl (600 mL). The organic phase was collected, then dried over anhydrous Na₂SO₄ and concentrated. 256 g off-white solid was obtained as the crude product, which was used in the next step directly. MS (ESI, m/e) [M+1]⁺ 579.0.

### Step 6: (S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid

To a solution of methyl(S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoate (105 g, 181.7 mmol) in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5˜6 with 3 N HCl acid at room temperature with stirring. The precipitate was filtered and dried in vacuum to give the product as a white solid (102.4 g, yield: 99%). ¹H NMR (400 MHz, DMSO-d6) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1H), 2.9-2.15 (m, 1H), 1.74-1.64 (m, 4H), 1.52-1.37 (m, 6H), 1.28-1.06 (m, 6H). MS (ESI, m/e) [M+1]⁺ 564.9.

### Step 7:2-((1H-pyrrolo[2,3-b]pyridin-5-yl)oxy)-N-((4-((((1r,4r)-4-hydroxy-4-methylcyclohexyl)methyl)amino)-3-nitrophenyl) sulfonyl)-4-(2-((S)-2-(2-isopropylphenyl) pyrrolidin-1-yl)-7-azaspiro[3.5]nonan-7-yl)benzamide (Sonrotoclax)

A mixture of (S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol) in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid was consumed completely, the reaction mixture was heated to ˜35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to room temperature, the precipitate was filtered and then the wet cake was washed twice with EA (180 mL). After drying in vacuum at 80-90° C., the desired compound was obtained (48 g, yield: 69.5%). ¹H NMR (DMSO-d6) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]⁺ 889.9.

What is claimed is:

1. A compound selected from:

| 1 | 2 | 3 | 4 |
|---|---|---|---|
| 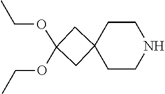 | 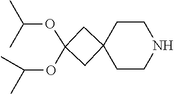 [SMILES: CC(C)OC1(OC(C)C)CC2(CCNCC2)C1] | 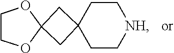 [SMILES: C1CC2(CCN1)CC1(C2)OCCO1] | 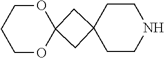 [SMILES: C1COC2(CC3(CCNCC3)C2)OC1] |

or a salt thereof.

2. The compound of claim 1, wherein the compound is 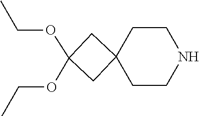
or a salt thereof.

3. The compound of claim 1, wherein the compound is 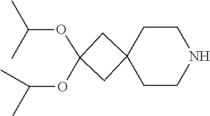 [SMILES: CC(C)OC1(OC(C)C)CC2(CCNCC2)C1]
or a salt thereof.

4. The compound of claim 1, wherein the compound is 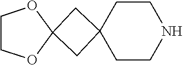 [SMILES: C1CC2(CCN1)CC1(C2)OCCO1]
or a salt thereof.

5. The compound of claim 1, wherein the compound is 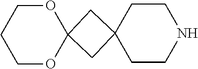 [SMILES: C1COC2(CC3(CCNCC3)C2)OC1]
or a salt thereof.

6. A method of making a compound of Formula (SII), 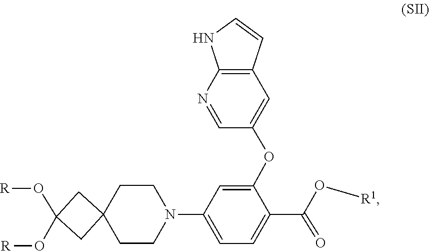
or a salt thereof, comprising reacting a compound of Formula (I): 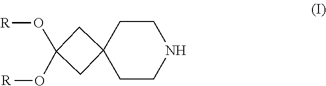
or a salt thereof with a compound of Formula (SI): 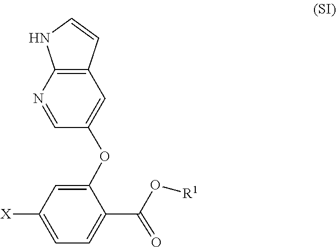
in the presence of a base,
wherein
at each of its occurrences, R is independently C₂₋₆alkyl or C₃₋₆cycloalkyl, wherein each of said C₂₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b}; or
two R together with the two oxygen atoms to which each is attached and the intervening carbon, form a 5- to 12-membered ring, said ring is optionally substituted with at least one substituent selected from halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of its occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl;
X is halogen; preferably, X is F, Cl, Br, or I; more preferably, X is F; and
R¹ is C₁₋₆alkyl, or C₃₋₆cycloalkyl, wherein each of said C₁₋₆alkyl or C₃₋₆cycloalkyl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —NO₂, —C(═O)R^{1a}, —C(═O)OR^{1a}, —OC(═O)R^{1a}, —OR^{1a}, —SO₂R^{1a}, —SR^{1a}, —NR^{1a}R^{1b}, —C(═O)NR^{1a}R^{1b}, —OC(═O)NR^{1a}R^{1b}, —NR^{1a}C(═O)NR^{1b}R^{1c} or —NR^{1a}C(═O)R^{1b};
at each of occurrences, R^{1a}, R^{1b} and R^{1c} are each independently selected from hydrogen, C₁₋₆alkyl, C₃₋₆cycloalkyl, -haloC₁₋₆alkyl or -haloC₃₋₆cycloalkyl.

7. The method of claim 6, wherein the base is 1,8-Diazabicyclo(5.4.0) undec-7-ene (DBU).

8. The method of claim 6 or 7, comprising reacting the compound of Formula (I) and the compound of Formula (SII) at a temperature between about 65° C. and about 75° C., or at a temperature of about 65° C., about 70° C., or about 75° C.

9. The method of any one of claims 6-8, further comprising reacting the compound of Formula (SII) with an acid or a base to provide a compound of Formula (SIII), 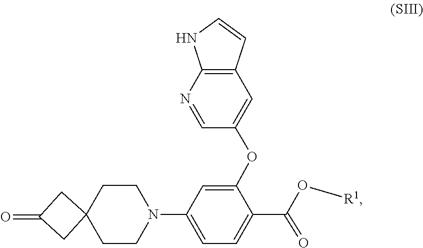
or a salt thereof.

10. The method of claim 9, wherein the acid is HCl, or the base is NaOH.

11. The method of claim 9 or 10, further comprising reacting the compound of Formula (SIII), or a salt thereof, with (S)-2-(2-isopropylphenyl) pyrrolidine, or a salt thereof, to provide a compound of Formula (SIV) 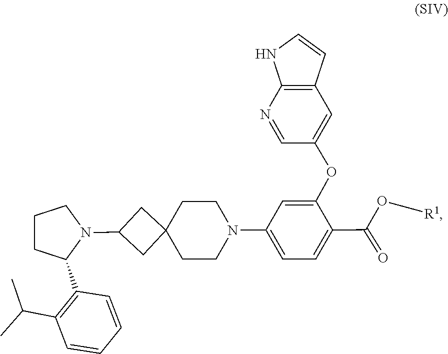
or a salt thereof.

12. The method of claim 11, comprising reacting the compound of Formula (SIII), or a salt thereof, with (S)-2-(2-isopropylphenyl) pyrrolidine, or a salt thereof, in the presence of NaBH(OAc)₃.

13. The method of claim 11 or 12, comprising reacting the compound of Formula (SIII), or a salt thereof, with (S)-2-(2-isopropylphenyl) pyrrolidine, or a salt thereof, at a temperature less than about 30° C.

14. The method of any one of claims 11-13, further comprising reacting the compound of Formula (SIV), or a salt thereof, with 4-((((1r,4r)-4-hydroxy-4-methylcyclohexyl)methyl)amino)-3-nitrobenzenesulfonamide, to provide Sonrotoclax, 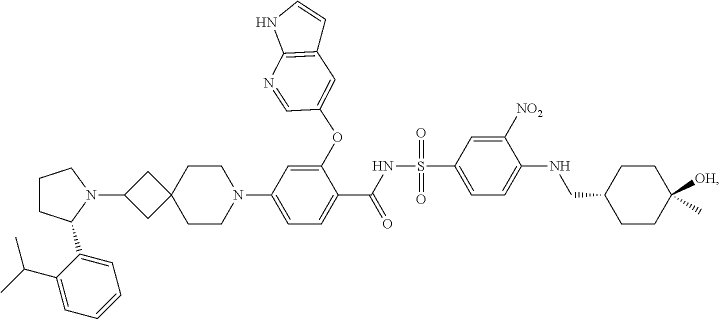 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof.

15. The method of claim 14, wherein the reaction happens in the presence of EDCI and DMAP.

16. The method of claim 14 or 15, further comprising reacting the compound of Formula (SIV), or a salt thereof, with an acid or a base, to provide (S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid 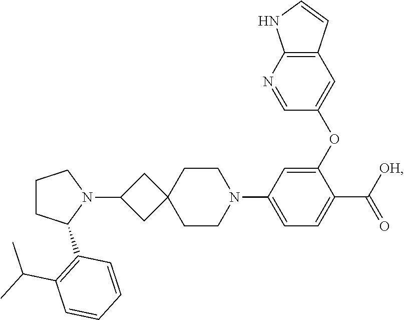 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof.

17. The method of claim 16, wherein the acid is HCl, or the base is NaOH.

18. The method of claim 16 or 17, further comprising reacting(S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid 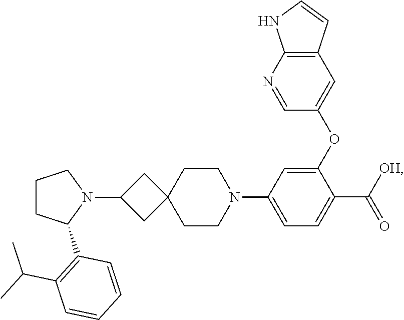 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof, with 4-((((1r,4r)-4-hydroxy-4-methylcyclohexyl)methyl)amino)-3-nitrobenzenesulfonamide, to provide Sonrotoclax, 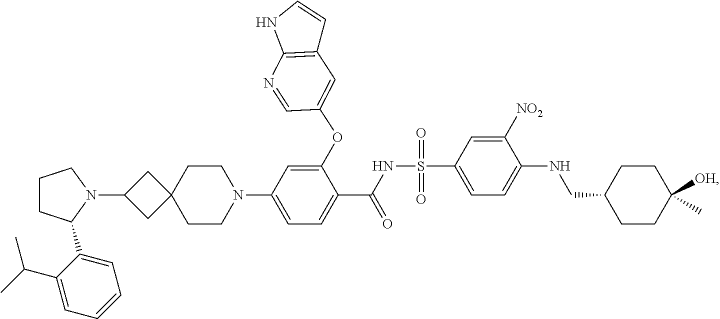 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof.

19. The method of any one of claims 16-18, comprising reacting(S)-2-((1H-pyrrolo[2,3-b]pyridin-5-yl)oxy)-4-(2-(2-(2-isopropylphenyl) pyrrolidin-1-yl)-7-azaspiro[3.5]nonan-7-yl)benzoic acid 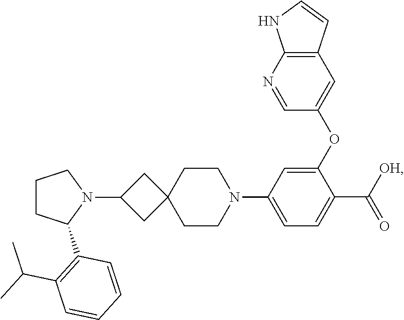 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof, with 4-((((1r,4r)-4-hydroxy-4-methylcyclohexyl)methyl)amino)-3-nitrobenzenesulfonamide in the presence of EDCI and DMAP.

20. A method for preparing a pharmaceutical composition comprising Sonrotoclax, comprising mixing Sonrotoclax with a pharmaceutically acceptable excipient, wherein Sonrotoclax is prepared according to the method of claim 14.

21. A method for preparing a pharmaceutical composition comprising Sonrotoclax, comprising mixing Sonrotoclax with a pharmaceutically acceptable excipient, wherein Sonrotoclax is prepared according to the method of claim 18.

22. A pharmaceutical composition comprising sonrotoclax or a pharmaceutically acceptable salt thereof and a pharmaceutically acceptable excipient, wherein sonrotoclax is prepared according to the method of any one of claims 6-19.
